(19) United States
(12) Patent Application Publication
(10) Pub. No.: US 2026/0144553 A1
(43) Pub. Date: May 28, 2026

(54) Suction Endoscopic Sheath and Assembly with Transposed Pressure Regulating Mechanism

(71) Applicant: SHAW P. WAN, CASTLE HAYNE, NC (US)

(72) Inventor: SHAW P. WAN, CASTLE HAYNE, NC (US)

(73) Assignee: Well Lead Global Limited, Tsuen Wan (CN)

(21) Appl. No.: 18/956,527

(22) Filed: Nov. 22, 2024

(51) Int. Cl. A61B 17/22; A61B 17/00; A61B 17/34

(52) U.S. Cl. CPC A61B 17/22; A61B 2017/00296; A61B 2017/22038; A61B 2017/22079; A61B 2017/3486

(57) ABSTRACT

A device for removing stones and foreign bodies from a human cavity including a straight sheath with one or more side branches; said straight sheath including a proximal tube and distal tube with respect to the operator who is holding the device; one or more side branches arise from the proximal tube and said branch or branches share a common lumen with the proximal and distal tubes; at the least, one of the side branches contains a pressure vent in the form of an opening that extends through the wall of the side branch and is sealed to form an airtight channel that extends toward the proximal tube. One or more openings at the end of this airtight channel serve as a transposed pressure regulating mechanism and are located and accessible on or near the surface of said proximal tube; an operator can regulate the aspiration pressure by covering or uncovering the vent or a portion of the vent.

References cited
US patent documents

| 10,828,050 B2 | Wan | November 2020 |
|---|---|---|
| 10,828,051 B2 | Wan | November 2020 |
| 2017/0,086,939 A1 | Vayser | March 2017 |
| 765,454,898 | Knapp | December 2013 |
| 2013/0,237,967 A1 | Schaeffer | September 2013 |
| 8,057,497 B1 | Raju | November 2011 |
| 2012/0143006 | Avitsian | June 2012 |
| 7,654,898 B2 | Knapp | February 2012 |
| 2010/0,204,712 A1 | Mallaby | August 2010 |
| 2009/0,216,246 A1 | Nita | August 2009 |
| 2008/0,188,831 A1 | Bonnette | August 2008 |
| 6,997,867 B2 | Soble | February 2006 |
| 6,159,195 | Ha | December 2000 |
| 5,971,938 A1 | Hart | October 1999 |
| 5,882,348 | Winterton | March 1999 |
| 2011/0,034,986 A1 | Chou | November 1998 |
| 5,830,224 | Cohn | November 1998 |
| 5,775,325 | Russo | July 1998 |
| 5,730,727 | Russo | March 1998 |
| 5,419,769 | Devlin | May 1995 |
| 4,534,542 | Russo | August 1985 |

Foreign Documents

| | | |
|---|---|---|
| WO2016/018,479 A1 | Wan | April 2016 |
| WO9,844,982 | Gordon | March 1998 |

### TECHNICAL FIELD

A surgical device and methods of using same to retrieve stones and other foreign bodies from a patient's body is disclosed herein.

### BACKGROUND ART

Flexible endoscopy has become a common surgical procedure. Flexible nephroscopes, ureteroscopes, and cystoscopes are the three scopes most commonly used in urology. They are often used in conjunction with stone fragmenting devices, lithotripters, and the procedures are collectively referred to as endoscopic lithotripsy.

A flexible endoscope generally comprises of two components, a handle and a shaft. The handle houses the deflection mechanism, and the shaft contains an imaging bundle and a working channel. To operate the flexible scope, the user generally uses the dominant hand to hold the handle and operate the deflecting mechanism and uses the thumb and the index finger of the non-dominant hand to stabilize the shaft. An endoscopic sheath is a conduit for the scope to enter a body cavity. It is generally a straight tube. To perform an endoscopic procedure irrigation is required. The irrigation is generally delivered with or through the scope. The out flow comes out between the sheath and the scope. When a conventional endoscopic sheath, such as a straight tube, is used, the outflow is passive. When the irrigation rate is higher than the space between the sheath and scope can handle then there will be accumulation of fluid in the body cavity, an undesirable event. When lithotripsy is performed using a flexible endoscope, for example when using the ureteroscope to remove a kidney stone, the stone fragments may come out between the scope and sheath; again, this passage of stone fragments is passive.

As a result, the suction sheath, as described in U.S. Pat. Nos. 10,828,050 B2 and 10,828,051 B2 has become very popular device. In the described devices, the outflow of fluid and stone fragments are active. Suction pressure is generally much greater than the irrigation rate; hence, fluid accumulation in the body cavity is not an issue. In addition, the outflow of the stone fragments is more efficient under suction. The suction sheath is generally comprised of a straight sheath and an oblique side branch. The side branch is to be connected to a suction apparatus. An air vent is often placed on this side branch serving as a pressure regulating mechanism to allow the operator to manipulate the aspiration pressure. The air vent on the side branch is important for its air and fluid dynamics.

### DISCLOSURE OF THE INVENTION

### Technical Problem

As mentioned previously, the operator needs to use one hand to hold the handle and at least two fingers of the other hand to stabilize the shaft; thus, making the manipulation of the air vent on the oblique side branch difficult (see FIG. 1). This often requires the surgeon to delegate the regulation of the pressure control mechanism to an assistant. The current disclosure relates to a device and method that solves this problem by transposing the air vent to the proximal segment of the straight shaft; thus, allowing the operator to manipulate the pressure regulating mechanism ergonomically using the remaining fingers in the hand that is stabilizing the endoscope shaft. Removing the air vent from the oblique side branch creates a problem with air flow that is resolved by structuring the sheath such that the air flows through the oblique side branch.

### Solution to the Problem

The current disclosure addresses numerous issues with the prior art and provides a means to more ergonomically manipulate the pressure regulating mechanism. This disclosure relates to a suction endoscopic sheath device and assembly for removing stones, stone fragments and other foreign bodies from a human body cavity. The device includes a straight sheath with one or more side branches. More specifically, the straight sheath includes a proximal tube and distal tube, with respect to the operator who is holding the device, and one or more side branches originating from the proximal tube. The one or more side branches share a common lumen with the straight sheath. In addition, at least one of the side branches contains a pressure vent in the form of an opening or set of openings extending through the wall of the side branch that is sealed or enclosed by an airtight pressure vent channel that extends toward the proximal tube of the sheath. An opening or set of openings in the pressure vent channel that is/are separate from the vent in the wall of the side branch and that are located on the side of the pressure vent channel that is opposite the side branch, serve as a transposed pressure regulating mechanism and is/are located and accessible on or near the surface or wall of the proximal tube such that an operator can regulate the aspiration pressure by completely or partially covering the vent or leaving the vent uncovered.

In some embodiments, an obturator with one or more central channels is inserted into the proximal end of the straight sheath, which is the same as the proximal end of the proximal tube. The distal end of the obturator extends beyond the distal end of the straight sheath, which is the same as the distal end of the distal tube, and the proximal end of the obturator can be releasably secured to the proximal tube of the straight sheath. In addition, a flexible water-tight seal with central aperture is secured to the proximal end of the straight sheath.

This disclosure also relates to suction endoscopic sheath assembly for removing stones, stone fragments and other foreign bodies from a human cavity including a straight sheath with one or more side branches and at least one of the side branches contains a pressure vent in the form of an opening, said opening extending through the wall of the side branch and is sealed to form an airtight pressure vent channel that extends toward the proximal tube of the straight sheath with one or more openings in a wall of the pressure vent channel that is/are located opposite to the side branch serving as a transposed pressure regulating mechanism. These openings are located and accessible on or near the surface of said proximal tube; an operator can regulate the aspiration pressure by covering and uncovering the vent or a portion thereof. An obturator with one or more central channels can be inserted into the proximal end of the straight sheath (proximal end of the proximal tube) and extends beyond the distal end of the said sheath (distal end of the distal tube), and the proximal end of the obturator can be releasably secured to the proximal end of the straight sheath. A flexible water-tight seal with central aperture can be secured to the proximal end of the straight sheath.

Other embodiments of the disclosed device and method of using same include a primary end of a primary tube releasably secured to the distal end of the oblique side branch, containing the transposed pressure regulating mechanism; and a distal end of the primary tube releasably secured to a collection container; and the proximal end of a secondary tube releasably secured to the collection container and the distal end of the secondary tube to a releasably secured to a negative pressure apparatus.

### BRIEF DESCRIPTION OF THE DRAWINGS

For the purpose of illustrating the invention, the drawings shown are in the forms that are presently preferred; it is understood, however, that this invention is not limited to the precise arrangements of the device as shown.

FIG. 1 is a schematic drawing of a left hand holding a conventional endoscopic suction sheath;

FIG. 2A-1 The perspective view of a preferred embodiment of the suction sheath for the left hand that uses a plug to close an opening in the pressure vent channel;

FIG. 2A-2 The perspective view thereof for right hand;

FIG. 2B-1 A perspective view of another embodiment of the suction sheath for the left hand that uses a push-pull valve to close an opening in the pressure vent channel;

FIG. 2B-2 A perspective view of thereof for the right hand;

FIG. 2C A cross-sectional view of the sheath cut along the longitudinal axis thereof;

FIG. 2D A cross-sectional view thereof cut along the transverse axis of the sheath;

FIG. 3A A perspective view of another embodiment of the suction sheath that uses a different (rotating) push-pull valve to close an opening in the pressure vent channel;

FIG. 3B A cross-sectional view of the sheath showing the pressure vent channel;

FIG. 3C is a cross-sectional view thereof cut along the transverse axis of the sheath;

FIG. 4A-1 is a front view of another embodiment of the suction sheath that uses a plug to close the opening in the pressure vent channel;

FIG. 4A-2 is a front view of another embodiment of the suction sheath that uses a push-pull valve to close the opening in the pressure vent channel;

FIG. 4B is cross-sectional view an embodiment cut along the longitudinal axis with no structure to close the opening in the pressure vent channel;

FIG. 4C-1 is a plan view of the same embodiment of the endoscopic suction sheath assembly;

FIG. 4C-2 is a cross-sectional view of the transposed pressure regulating mechanism cut along line A-A in FIG. 4C-1;

FIG. 4D is a front plan view of another embodiment of the suction sheath that includes a detachable and tearable distal tubes;

FIG. 5A is a cross-sectional view of one single channel embodiment of the obturator with receptacle;

FIG. 5B is a cross-sectional view of the double channel embodiment of the obturator each with its own receptacle;

FIG. 6A is a cross-sectional view of an embodiment of the assembly with a deflectable tip;

FIG. 6B is a plan view thereof;

FIG. 7A is a front perspective view of the embodiment with a balloon assembly;

FIG. 7B is a side perspective view thereof;

FIG. 8A is a front perspective view of an embodiment with accessory side arm;

FIG. 8B is a side perspective thereof;

FIG. 9A is a front perspective view of an embodiment with a concentric distal tube;

FIG. 9B is a cross-sectional view thereof cut along the longitudinal axis;

FIG. 9C is a cross-sectional view thereof cut along the transverse axis of the concentric distal tube;

FIG. 10A is a front perspective view of the embodiment of the assembly that includes an accessory side arm with an independent channel;

FIG. 10B is a cross-sectional view thereof cut along the longitudinal axis;

FIG. 10C is a cross-sectional view thereof cut along the transverse axis of the distal tube;

FIG. 11A is a perspective view of a needle shaft for use with the obturator;

FIG. 11B is a perspective view of the needle sheath for the needle assembly;

FIG. 11C is a perspective view of the assembled needle assembly;

FIG. 11D is the assembled needle, obturator assembly;

FIG. 11E is the assembled needle assembly inside the obturator which in turn is inserted into the suction endoscopic sheath, the needle/obturator/sheath assembly; and

FIG. 12 is the full suction endoscopic sheath assembly with transposed pressure regulating mechanism

### MODES FOR CARRYING OUT THE INVENTION

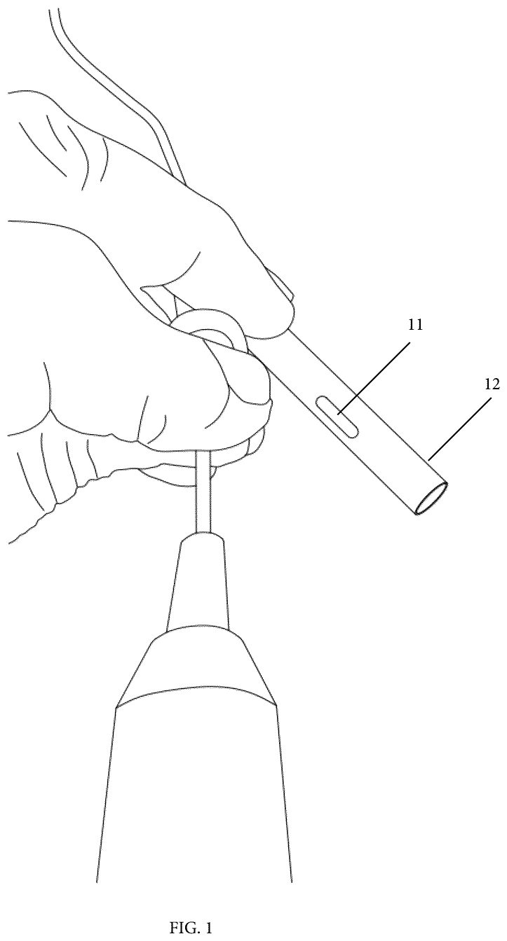

FIG. 1 shows a version of the endoscopic suction sheath assembly that is currently used. There is an opening 11 in a side branch 12 that is difficult for the user to reach while using the left hand to hold the sheath 10 and the right hand to hold the endoscope that is inserted through the sheath.

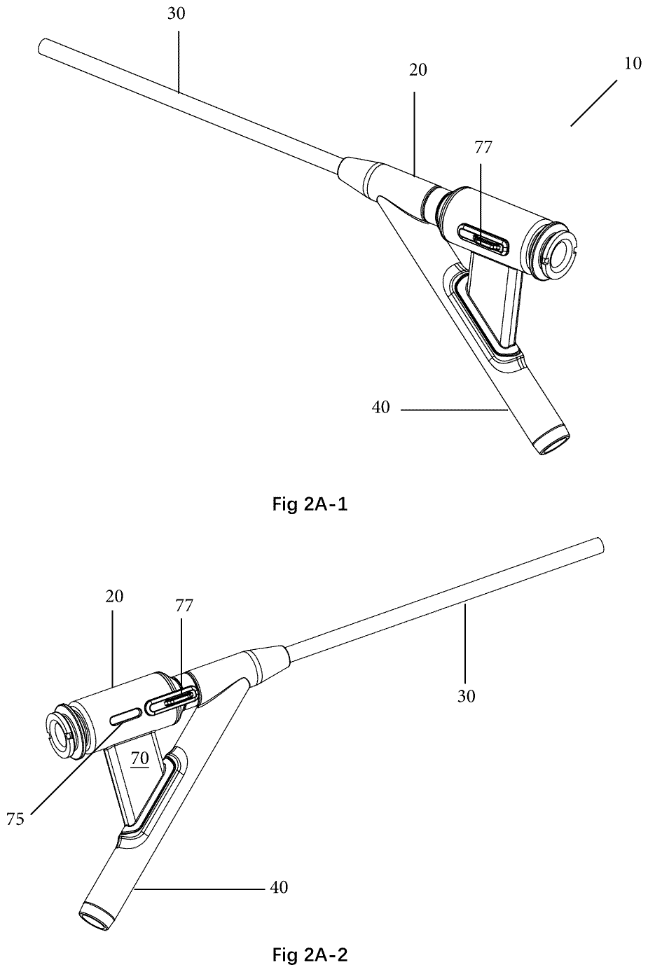

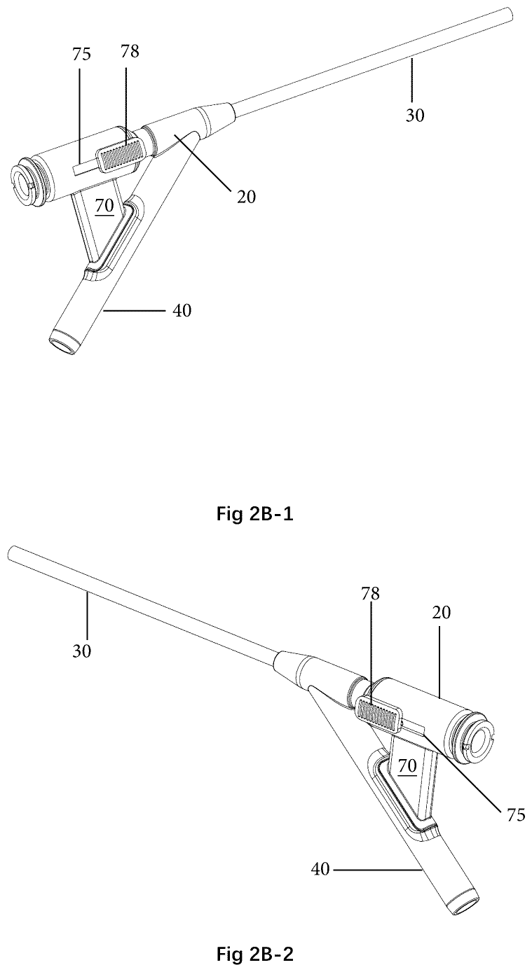

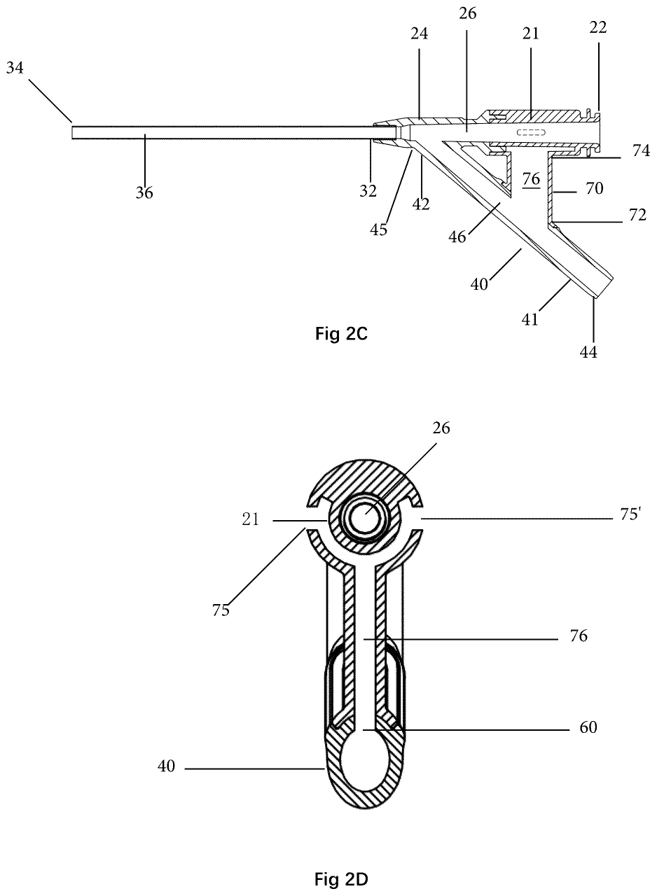

FIGS. 2A-1 and 2A-2 show a preferred embodiment of the endoscopic sheath. FIGS. 2B-1 and 2B-2 show an alternate embodiment of the same suction sheath. FIGS. 2C and 2D are cross-sectional views that illustrate a basic embodiment of the suction sheath 10. FIG. 2C is cut along the longitudinal axis of the sheath 10 and FIG. 2D is cut along the transverse axis of the sheath 10.

Each embodiment has a sheath 10 includes a proximal tube 20 and a distal tube 30 with respect to the operator who is holding the endoscopic sheath 10. The proximal tube 20 has a proximal end 22 and distal end 24, a lumen 26 and a wall or surface 21. The distal tube 30 has a proximal end 32 and distal end 34, a lumen 36 and a wall or surface 31. The lumen 26 of the proximal tube 20 and the lumen 36 of the distal tube 30 communicate with each other or are otherwise continuous with each other. Distal tube 30 may have graduate markings 38. The proximal tube 20 and the distal tube 30 can be constructed as a single piece or two pieces releasably or non-releasably joined together. The material to produce the tubes can be rigid, semi-rigid, or flexible. In the preferrable embodiment, the proximal tube 20 is made from material that is rigid and the distal tube 30 is made from any biocompatible material that is semi-rigid or flexible.

Some embodiments of the distal tube are uniformly flexible along their length, i.e. from the proximal end 32 to the distal end 34. Some embodiments of the distal tube 30 are not uniformly flexible, but rather can increase or decrease flexibility gradually and uniformly as the tube 30 extends from the proximal end 32 to the distal end 34.

Radiopaque material may be added to enhance its visualization under x-ray. The surface of the sheath 10, especially the distal tube 30, can be coated with a combination of hydrophilic, anti-microbial, anti-encrustation, or radiopaque coatings. Sheath 10, especially the distal tube 30, can be reinforced with coil to add structure and integrity to allow the tube 30 to withstand suction and to change rigidity/flexibility. The coil can be metal, plastic, or alloys. Sheath 10 can be any color and opacity. In the preferred embodiment, the proximal tube 20 is transparent, and the distal tube 30 is opaque.

Each embodiment has one or more oblique side branches 40 that extend away from the distal end 24 of the proximal tube 20. Each of the at least one side branches 40 has a proximal end 42 and distal end 44 with respect to the proximal tube 20. Each side branch 40 also has a lumen 46 extending through the side branch 40 from the proximal end to 42 the distal end 44 that communicates with the lumens 26 and 36 of the proximal tube 20 and distal tube 30. The side arm 40 extends away from the distal end 24 of the proximal tube 20 at an angle to a longitudinal axis of the proximal tube 20 that is greater than 0 degrees and less than 180 degrees. In some preferred embodiments, the angle is between 35 to 50 degrees toward the proximal end 22 of the proximal tube 20 of the sheath 10. The distal tube 30 is constructed with the same or different material as the proximal tube 20. The proximal end 42 of the side branch 40 can be releasably, i.e. through a male-female screw type arrangement, or non-releasably joined as manufactured with a single injection mold with the proximal tube 20 of the sheath 10. The adjoining point is shown as 45. In the preferred embodiment the oblique side branch 40 is non-releasably attached to the proximal tube 20 of sheath 10 and is manufactured using the same biocompatible transparent plastic as the proximal tube 30 of the sheath 10.

The side branch 40 also includes a pressure vent 60 located on this side branch. The pressure vent 60 is an opening the extends through the wall 41 of the side branch 40 and is in direct communication with the lumen 46 of the side branch 40. The opening 60 can be any shape or form. In preferred embodiments, the pressure vent 60 is a longitudinal slit approximately 12 mm×1.5 mm in size.

The pressure vent channel 70 is a structure or wall that encloses the pressure vent 60. Channel 70 has a proximal end 72 and distal end 74 with respect to the pressure vent 60. That proximal end 72 encases the pressure vent 60 and the distal end 74 extends to the proximal tube 20 adjacent to the proximal end 22. Channel 70 has a lumen 76, and an opening or openings 75, 75′ which is/are located near the surface of the proximal tube 20 near the proximal end 22. An alternate embodiment has a “free-standing” channel 70 that consists of a tube that connects at one end to the pressure vent 60 and a second, opposite end that does not connect any portion of the sheath 10. The opening serves as a transposed pressure regulating mechanism. To regulate the suction pressure, the opening 75 can be covered, partially covered, or left open by using the operator's finger or by a plug or a push-pull, a rotary, or some other type of valve that can cover, partially cover, or uncover the opening 75. The channel 70 may releasably or non-releasably attached to the side arm 40 where it encloses the pressure vent 60. The channel 70 may be constructed with rigid, semi-rigid non-collapsible, or flexible non-collapsible biocompatible material. It can be manufactured with the same or different material as the side branch 40. In a preferred embodiment the channel 70 non-releasably attaches to the side branch 40 and thereby encloses the pressure vent 60 and is manufactured with the same material as the oblique side branch 40.

The lumen 46 of the oblique side branch 40 communicates with the lumen 26 of the proximal tube 20 at the adjoining point 45 where the proximal end 42 of the side branch 40 meets the distal end 24 of the proximal tube 20. Lumen 76 communicates with lumen 46 at the point of the pressure vent 60. However, lumen 76 does not communicate directly with lumen 26, but instead extends partially or completely around a portion of the surface 21 of the proximal end 22 of the proximal tube 20. Alternately, the lumen 76 of the channel 70 can connect the pressure vent 60 at one end and extend away from the pressure vent 60 toward proximal end 22 of the proximal tube 20 without connecting to another structure in the device.

In a preferred embodiment and the inventor's anticipated best mode, the opening 75 extends parallel to the longitudinal axis of the proximal tube 20 and is preferably approximately 1.5 mm in width and 15 mm in length. The pressure vent channel 70 can be non-releasably attached to the surface 21 of the proximal end 22 of the proximal tube 20 of the sheath 10, such as manufacturing as a single entity, or as stand-alone component resting on the surface of the proximal end 22 of the proximal tube 20 of the sheath 10.

In one of the preferred embodiments, the pressure vent channel 70 extends onto and encloses or encircles at least part of the surface 21 of the proximal end 22 of the proximal tube 20 in a semicircular or circular fashion. These embodiments can have multiple pressure vents 75, 75′ on either side of the surface of the proximal tube 20. (see FIG. 2D) Both vents may have a free-standing (separable) or attached plug 78 (see FIGS. 2A-1 and 2A-2) or a push-pull valve 78 (see FIGS. 2B-1 and 2B-2) to seal the openings 75, 75′. This configuration allows the operator to select various functions: left hand holding, right hand holding, one vent completely covered, other vent covered, partially covered, or left open, or a number of other combinations.

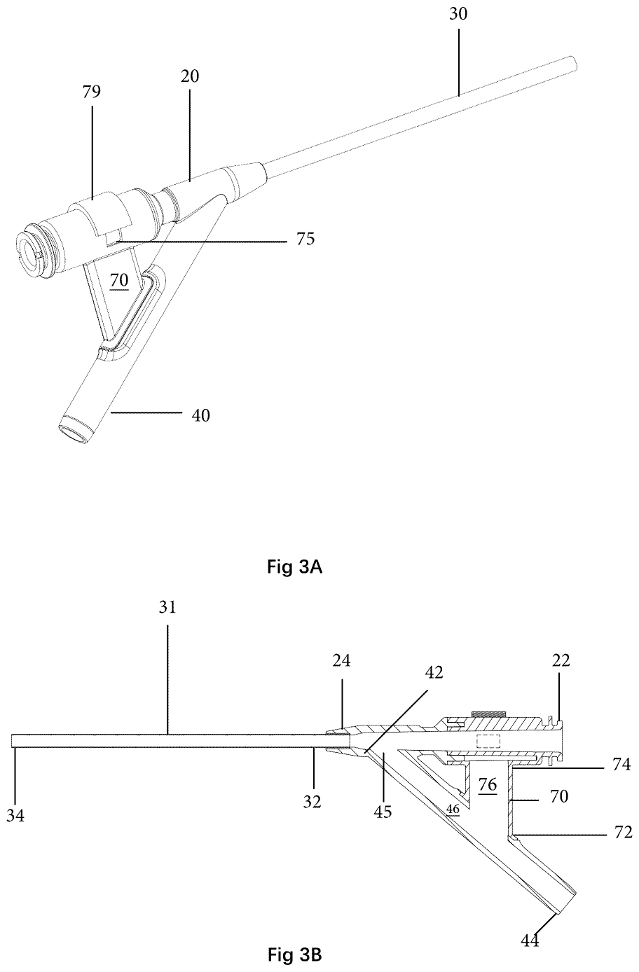

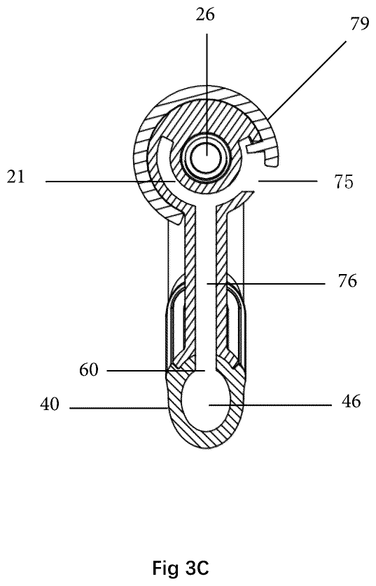

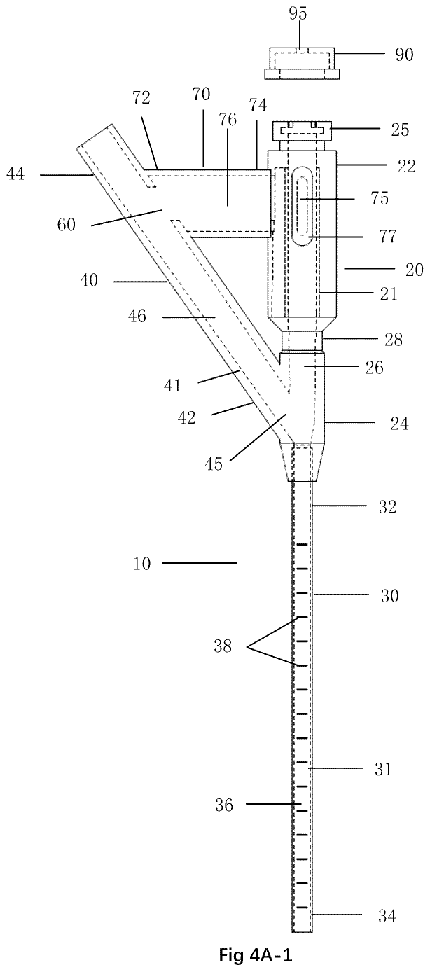

FIGS. 3A-3C show an alternate embodiment of the same sheath 10. In this embodiment, the channel 70 extends partially around the surface of the proximal end 22 of the proximal tube 20 on one side with an opening 75 on the ipsilateral side of the proximal tube 20. The opening 75 may have a rotary push-pull valve 79, to allow the operator to cover the opening 75 completely, partially, or not at all.

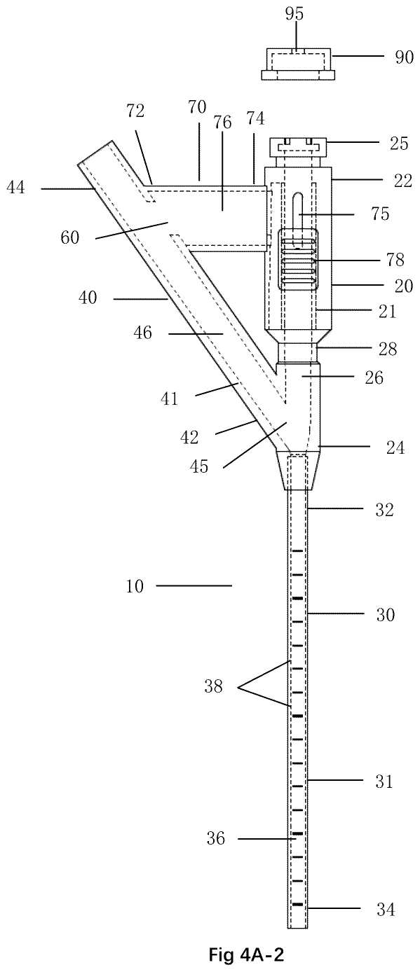

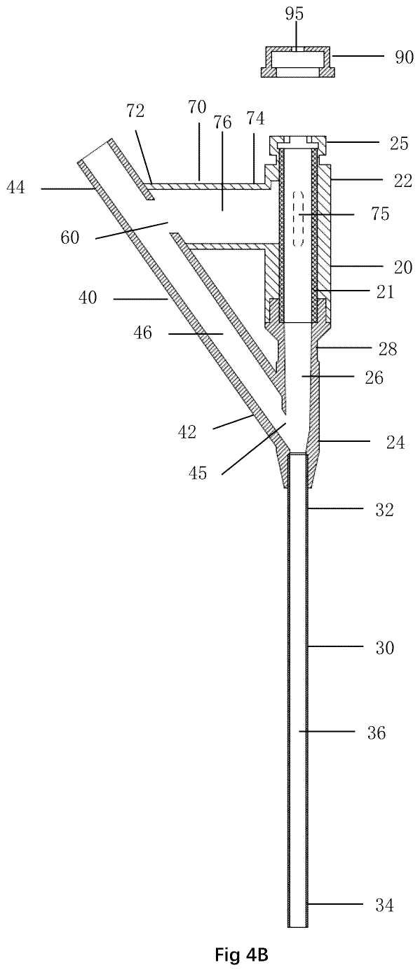

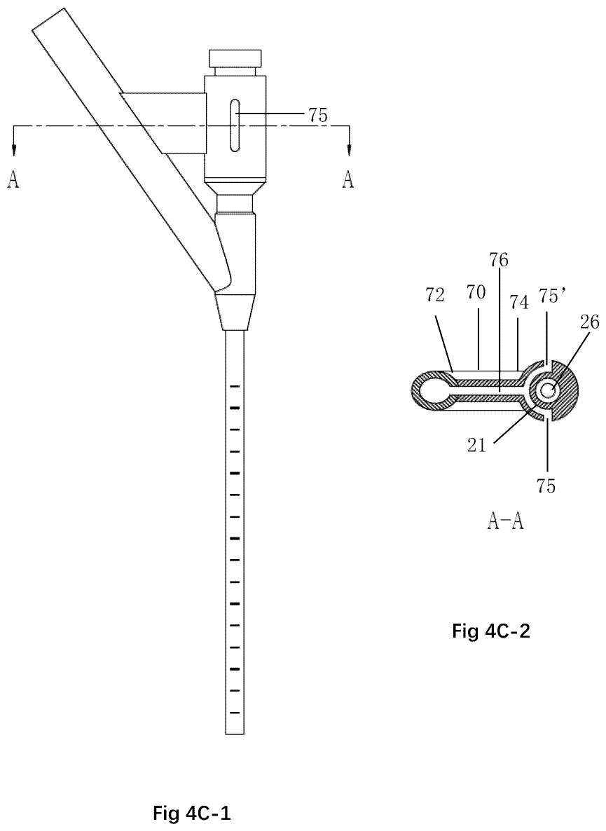

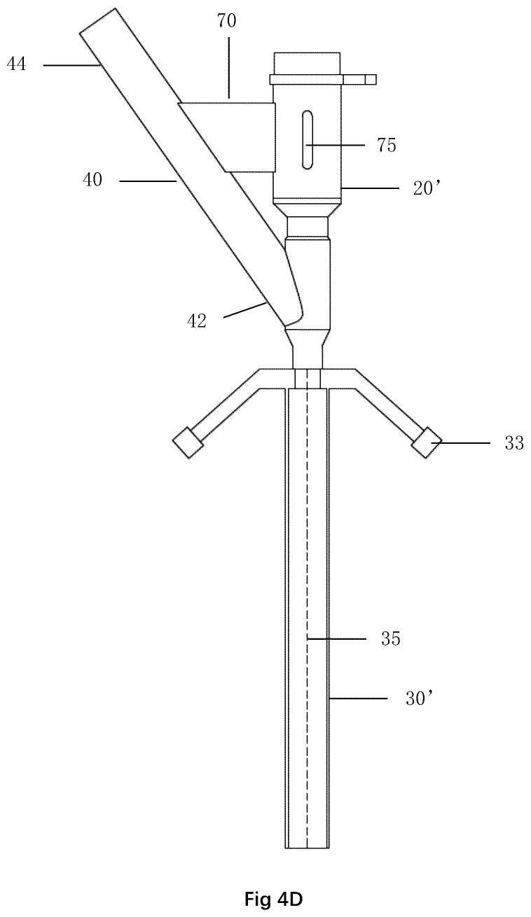

FIGS. 4A-4D show the details of the embodiments of the sheath 10 shown in FIGS. 2A-1 through 2B-2. As shown in FIG. 4C-2, which is a cross-sectional view of the sheath 10 cut along the line labeled A-A in FIG. 4C-1, the lumen 76 of the channel 70 extends to and/or around the surface 21 of the proximal end 22 of the proximal tube 20 in a semicircular fashion with two openings 75 and 75′ on either side of the proximal tube 20. Both vents 75 and 75′ may have a free-standing or an attached plug (see FIG. 4A-1) to seal the vents 75, 75′. This configuration allows the operator to select various functions such as left hand holding, right hand holding, one vent completely covered, other vent completely covered, partially covered or left open, or a number of other combinations.

In the embodiments shown in FIG. 4D, a releasable distal tube 30′ can be split longitudinally away from the rest of the device; thus facilitating the insertion of an indwelling Foley catheter through the distal tube 30′, as the Foley catheter has an inflation port that cannot be disengaged from a straight tube once the balloon has been inflated. This tearable tube can be manufactured with a perforated line 35 that allows splitting of the distal tube away from the rest of the device or using a tearable or perforated material with two flanges 33 where the operator can grasp the flanges and tear the releasable distal tube (30′) away from the proximal tube (20).

In another embodiment shown in FIG. 4A-2, one or both openings 75 and 75′ may have a push-pull valve 78 to allow operator to select various functions: left hand holding, right hand holding, one vent completely covered, other vent covered, partially covered, or left open, or a number of other combinations. The push-pull valve 78 in this embodiment is a switch-like structure that can be moved back and forth within the openings 75 and/or 75′. As seen in FIGS. 4A and 4B, color band 28 is placed at a location just proximal to adjoining point 45 with the side branch 40 to demarcate the orifice or opening to the lumen 46 of the side branch 40. FIGS. 4A and 4B also show a flexible seal or cap 90 that is made of biocompatible flexible material such as but not limited to rubber or silicone. It is releasably or non-releasably secured to the proximal end of proximal tube 20. The flexible cap 90 has a central aperture 95 which allows passage of an endoscope or other instrument and at the same time maintains an air- and water-tight seal.

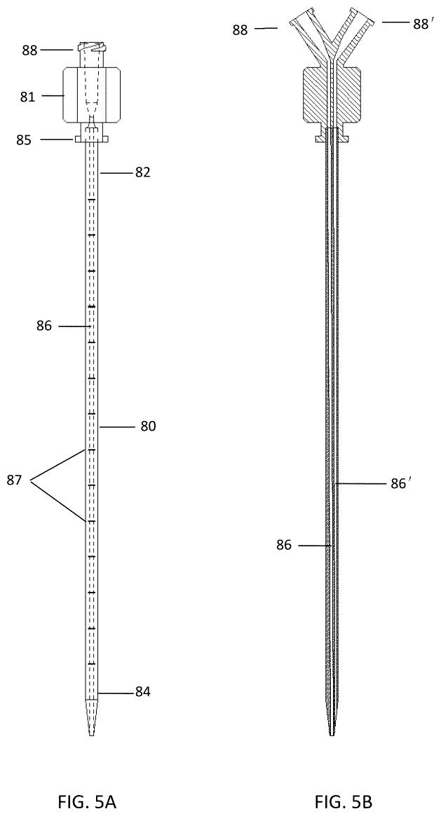

FIGS. 5A and 5B show Obturator 80 has a proximal end 82 and distal end 84 with respect to the operator who is holding the device. A handle 81 is incorporated into the proximal end 82. Distal end 84 may be tapered or round, pointed or blunt. The obturator 80 is longer than the sheath 10 with the distal end 84 extending beyond the distal end 34 of the distal tube 30 of the sheath 10 when the obturator 80 is inserted into the proximal end 22 of the proximal tube 20. The proximal end 82 is releasably engaged with the proximal end 22 of proximal tube 20 of the sheath 10 through a locking mechanism such as male-female luer lock (25, 85). Obturator 80 has one or more (86, 86′) central channels. The proximal end 82 of the obturator 80 has a receptacle 88 for the single channel 86 that allows insertion or attachment of a device such as a guide wire, a syringe, a stone basket, a scope, or irrigation tubing. As shown in FIG. 5B, some embodiments have two or more channels 86, 86′, one channel 86 can be used for one device or function, and a second, separate channel 86′ can be used for an additional function or device. In these embodiments, each channel 86, 86′ has its own receptacle (88,88′) that allows insertion or attachment of a device such as a guide wire, a syringe, a stone basket, a scope, or irrigation tubing. Obturator 80 can be constructed with any biocompatible material and can be rigid, semi-rigid or flexible. Radiopaque material can be added to increase its visibility under x-ray. There may be graduate markings 87 on the surface of the obturator 80. Obturator 80 can be coated with a combination of hydrophilic, anti-microbial, or anti-encrustation coatings.

Flexible, water-tight seal 90 is made of biocompatible flexible material such as but not limited to rubber or silicone. It is releasably or non-releasably secured to the proximal end 22 of proximal tube 20. The flexible cap has a central aperture 95 which allows passage of an endoscope or other instrument at the same time maintains air- and watertight seal.

### Deflectable Tip

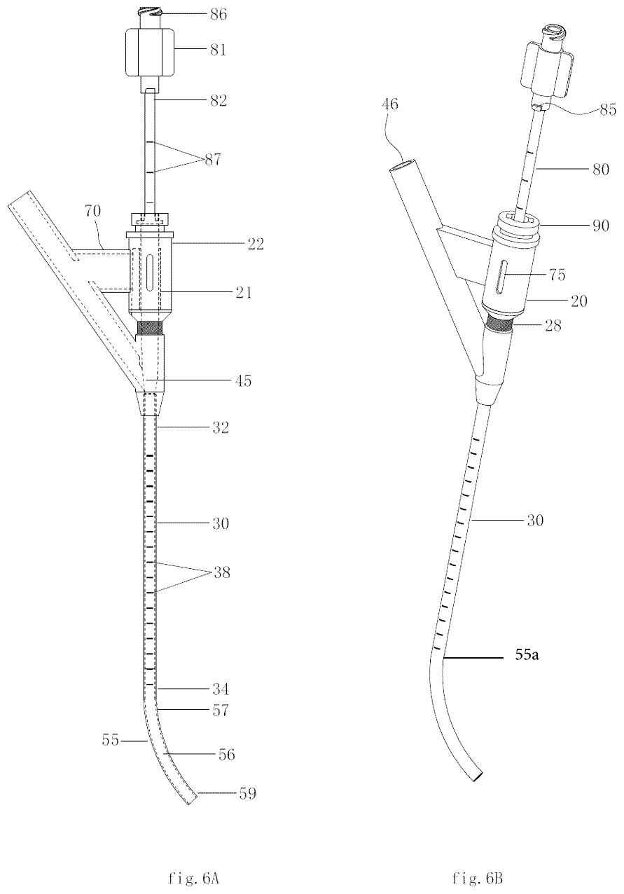

FIGS. 6A and 6B show an alternate embodiment of the endoscopic sheath that incorporates a flexible, deflectable tip 55. In preferred embodiments, the tip 55 includes a transition point 55a where the flexibility of the deflectable tip 55 increases to allow passive or active deflection. In this embodiment, the distal end 34 of the distal tube 30 includes a flexible tip 55 with a proximal end 57, distal end 59 and a lumen 56. The deflectable tip 55 will enable the user to adjust the direction of suction, irrigation, instrument placement, or removal of a stone, stone fragment or any other foreign body or tissue from a patient. Lumen 56 should be the same size or smaller than lumen 36 of the distal tube 30 of the sheath 10. The flexible tip has a length in the range of, but is not limited to, 2 to 12 cm. The flexible tip 55 is deflectable to any angle from 0 to 360 degrees as needed by the user of the device. The deflectable tip 55 of the distal tube 34 can be deflected either passively or actively. Passive deflection depends on the innate active deflection mechanism of an instrument such as a flexible scope or a deflectable obturator inside the sheath 10. In the passive mode, the deflectable tip 55 of the distal tube 34 has an inherent bending range up to 360 degrees. In the active mode, the distal tube 30 is straight. A cable or some other mechanism can be either withdrawn or shortened gradually thus bending the distal portion 59 of the deflectable tip 55 to a maximum of 360 degrees. The flexible tip 55 may also have memory which is to say that the flexible tip 55 may be configured into a specific angle or form such that it retains that form. It can be inserted into the body in one configuration, i.e. straight, using an obturator 80 or scope until it is manipulated into a specific location by the user of the device. The flexible tip can then be reverted back to the original shape by withdrawing the obturator or scope. The deflectable tip 55 may be constructed with the same material as the distal tube 30 of the sheath 10 or can manufactured with a different biocompatible material. It can also be reinforced with inner coil. In some embodiments, the flexible tip 55 is not uniformly flexible and the flexibility of the deflectable tip 55 may increase or decrease as one moves from the proximal end 57 to the distal end 59 of the tip 55. In preferred embodiments, the flexibility of the deflectable tip 55 is not uniform. The transition in flexibility from distal tube 34 to proximal end 57 of deflectable tip 55 is not gradual; rather, there is a transition point 55a where the deflectable tip 55 becomes considerably more flexible than the distal end 34 of the distal tube 30. The distal end 59 of the flexible tip 55 is progressively more flexible than the proximal end 57. It is the distal end 59 of the flexible tip that needs to be more flexible than the rest of the distal tube 30.

### Balloon Assembly

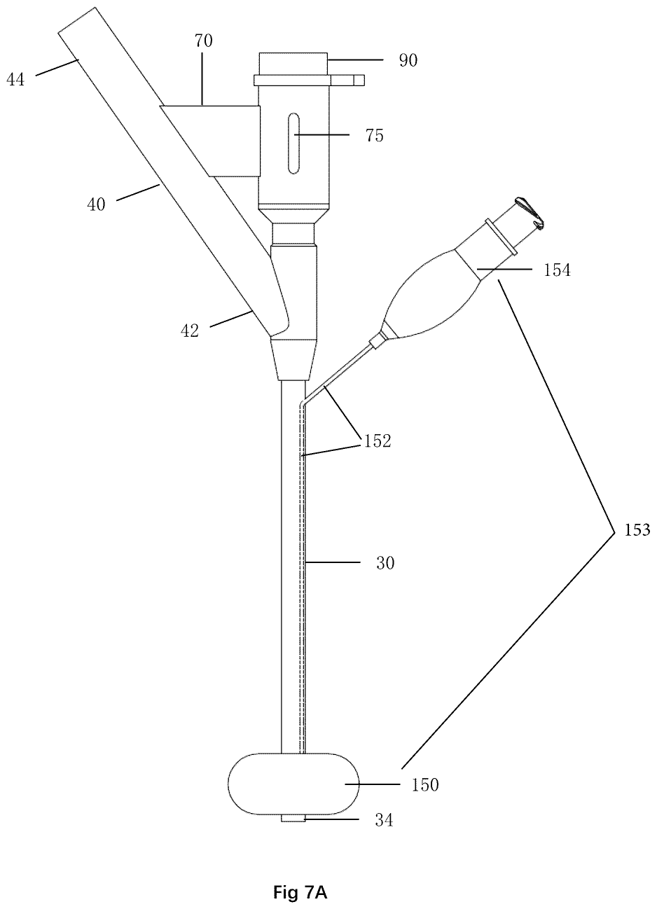

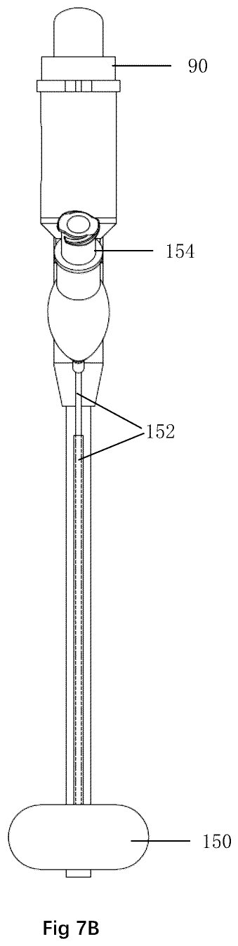

FIGS. 7A-7B illustrate another embodiment that incorporates a balloon assembly 153 into the endoscopic sheath assembly 10. A balloon assembly 153 is operationally associated with the distal end 34 of the distal tube 30 or the distal end 59 of the deflectable tip 55 of the suction sheath 10. When the suction sheath 10 is inserted into a patient and placed in a desired location, the balloon 150 is inflated to hold the sheath 10 in place. This is especially useful when it is placed within the lumen or cavity of a patient's body. As illustrated, a balloon 150 which is located at the distal end 34 of the distal tube 30 or 59 of the deflectable tip 55 of the sheath 10. A balloon inflation channel 152 emanates off of the balloon inflation valve 154, creating a pathway from the balloon inflation valve 154 to the balloon 150 such that passing gas or liquid through the balloon inflation valve 154 and the channel 152 causes the balloon 150 to inflate.

### Secondary Side Arm(s)

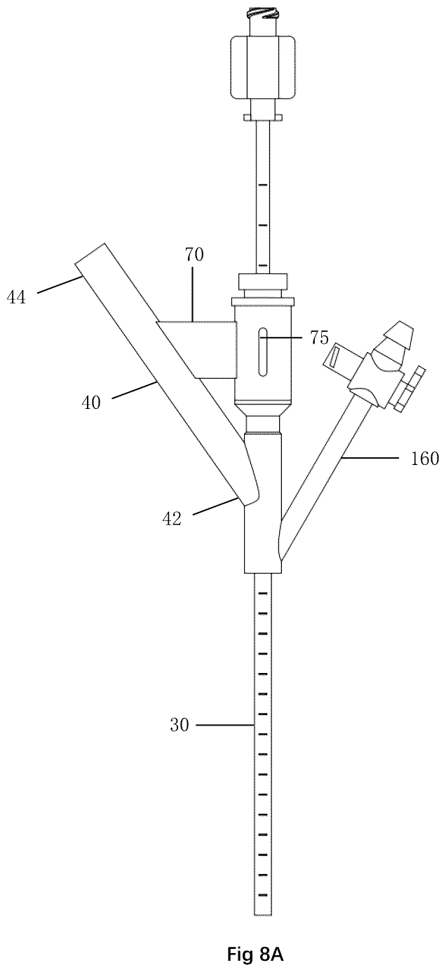

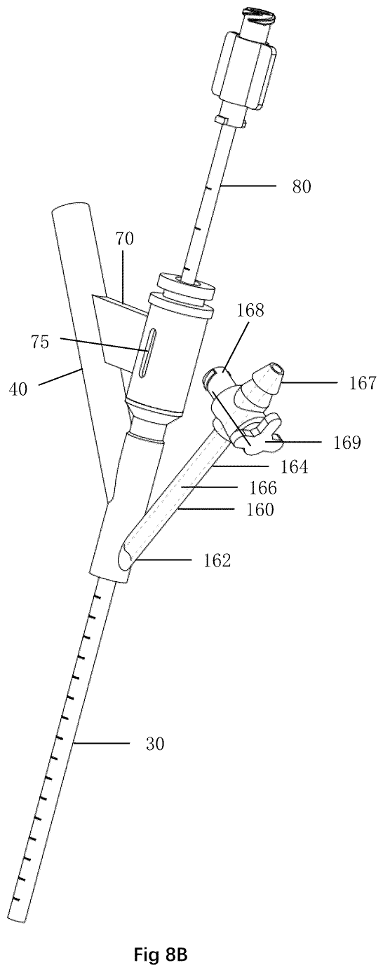

FIGS. 8A and 8B illustrate yet another embodiment of the endoscopic sheath assembly 10 that includes an accessory side arm 160. The accessory side arm 160 emanates from the surface 21 of the proximal tube 20 at an angle of between 0° and 180°. In preferred embodiments, the accessory side arm 160 extends away from the outer surface 21 of the distal end 24 of the proximal tube 20 at an oblique angle to the longitudinal axis of the sheath 10. In some embodiments the accessory side arm 160 is located distal to the side branch 40. The accessory side arm 160 has a lumen 166 which communicates with the lumens 26, and 36, (and 56 when the flexible tip 55 is present) of the sheath 10. The accessory side arm 160 has a proximal end 162 and distal end 164, with respect to the proximal tube 20. The distal end 164 may have one or more sluices 167,168 through which additional instruments or irrigation devices can be passed through. A valve 169 may be incorporated with the distal end 164 allowing the operator to choose the desired sluice for the passage of additional instruments or irrigation. Some embodiments may have a plurality of accessory side arms 160 each with its own lumen that communicates with one or more of the other lumens 26, 36, and 56 present in or associated with the sheath 10.

### Second Distal Tube

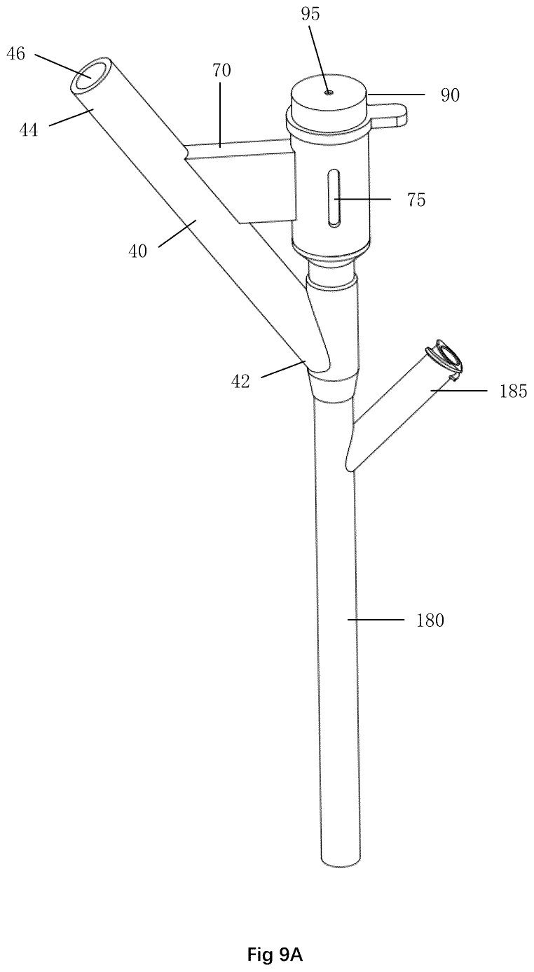

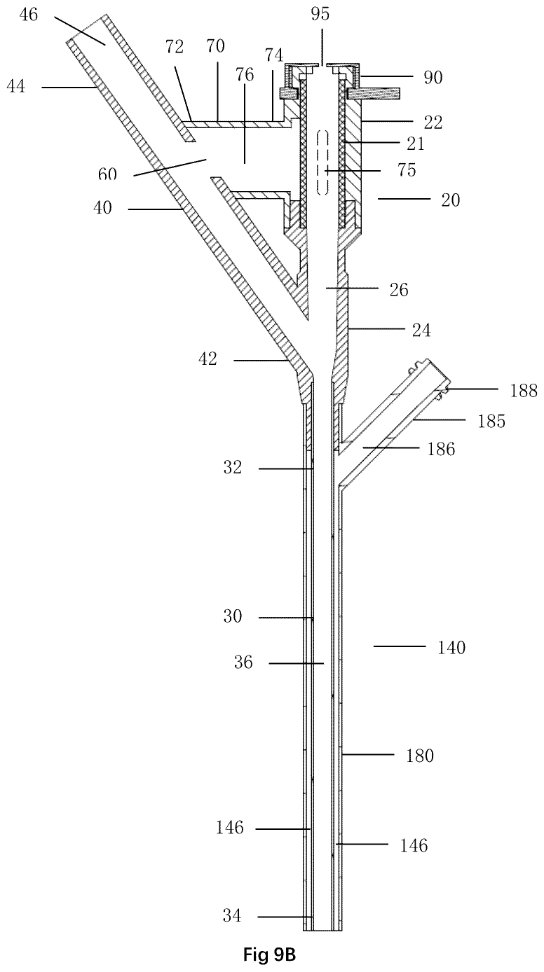

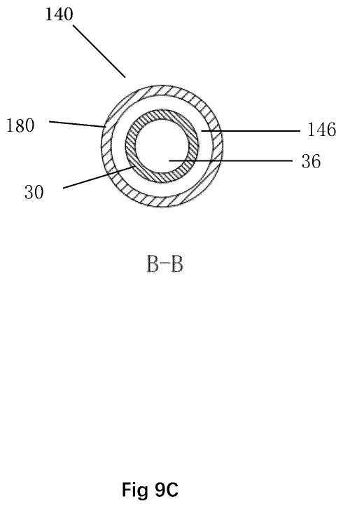

FIGS. 9A-9C illustrate yet another embodiment of the sheath assembly 10 that incudes concentric tubes. More specifically, in these embodiments, the distal tube 140 is formed by two concentric tubes, a second distal tube (180) that is larger than the first distal tube (30). The second distal tube 180 detachably, i.e. through a male-female luer lock, or non-detachably encloses the first distal tube 30 concentrically and also encloses a second lumen 146. The lumen (36) of the smaller tube (30) is continuous with and/or communicates with the distal end (24) and lumen (26) of the proximal tube 20 of the sheath 10 and the lumen (46) of the first side arm (40). The lumen (146) of the larger tube (180) is continuous with and/or communicates with a lumen (186) of a second side arm (185). In essence this configuration forms two separate channels for the distal tube (140). The distal end of the second side arm (185), with respect to the distal tube (140) may be constructed with a receptacle such as a luer lock (188) to allow the attachment of an instrument or irrigation tubing. The two concentric tubes (30, 180) can be constructed with the same biocompatible materials or different materials. Each tube may also have its own rigidity.

### Additional/Accessory Side Arm(s)

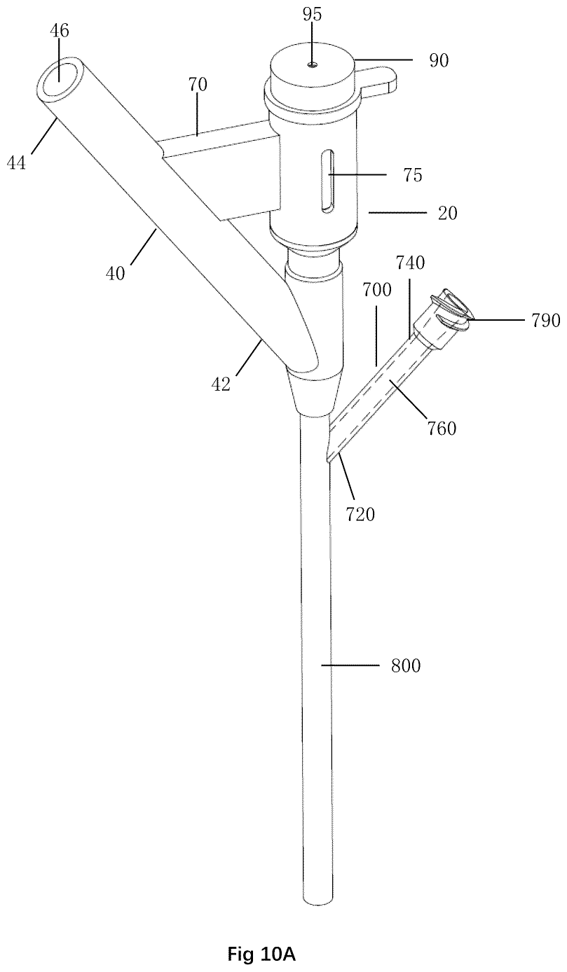

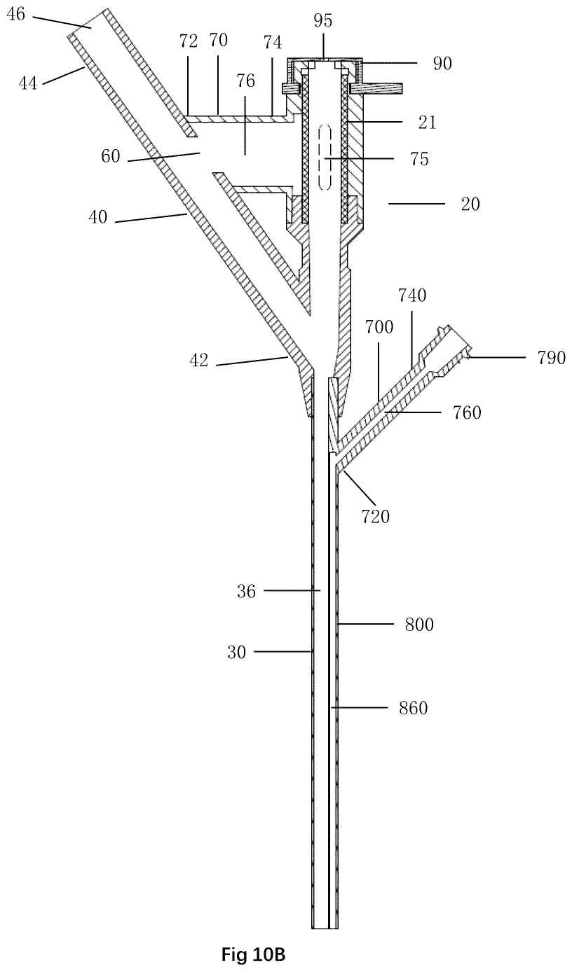

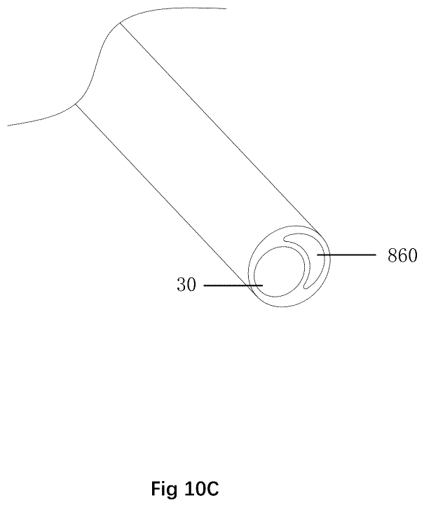

FIGS. 10A-10C illustrate yet another embodiment of the endoscopic sheath that includes an accessory side arm with an independent channel. In these embodiments, a secondary tube 800 is secured, non-concentrically, to an outer surface of the distal tube 30 of the sheath 10, and in at least one embodiment the secondary tube 800 has a side arm 700. Side arm 700 has a proximal end 720 and a distal end 740, with respect to the tube 800, and a lumen 760. The lumen 760 of the accessory side arm 700 is continuous with and communicates with an accessory channel 860 of the tube 800 and is separate and independent of the lumens 26,36 of the proximal tube 20 and the distal tube 30 of the sheath 10.

In yet another embodiment, there are two secondary side arms 700, 701 that are secured to the outer surface 31 of the distal tube 30 of the sheath 10; each secondary tube 800 has its own side arm 700 and channel or lumen 760 that is independent of the lumen 36 of the distal tube 30 of the sheath 10. The distal end 740 of the second side arm 700 may be constructed with a receptacle such as a luer lock 790 to allow attachment of an instrument or irrigation tubing. The side arm(s) 700 of the secondary tube 800 may be used to guide additional irrigation or to insert an instrument or device through its lumen(s).

### Needle Assembly

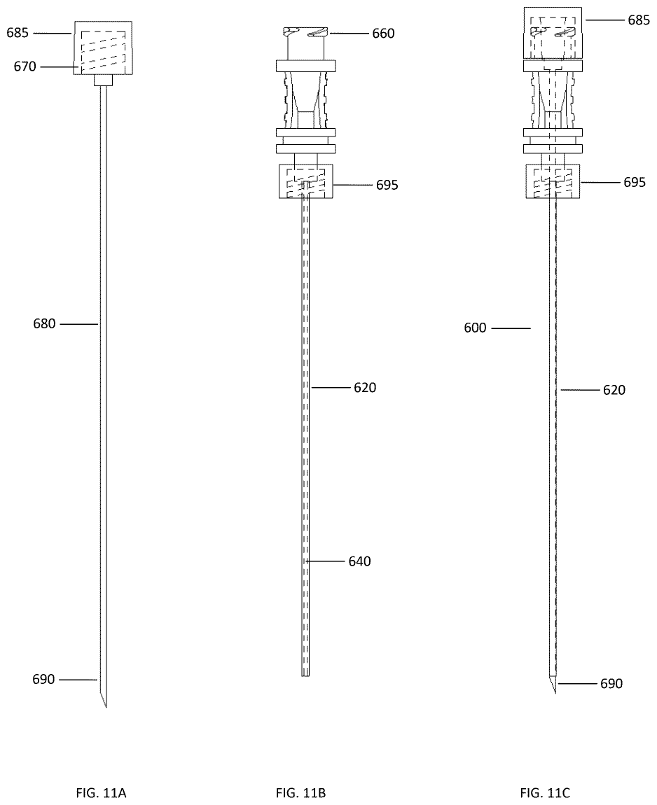

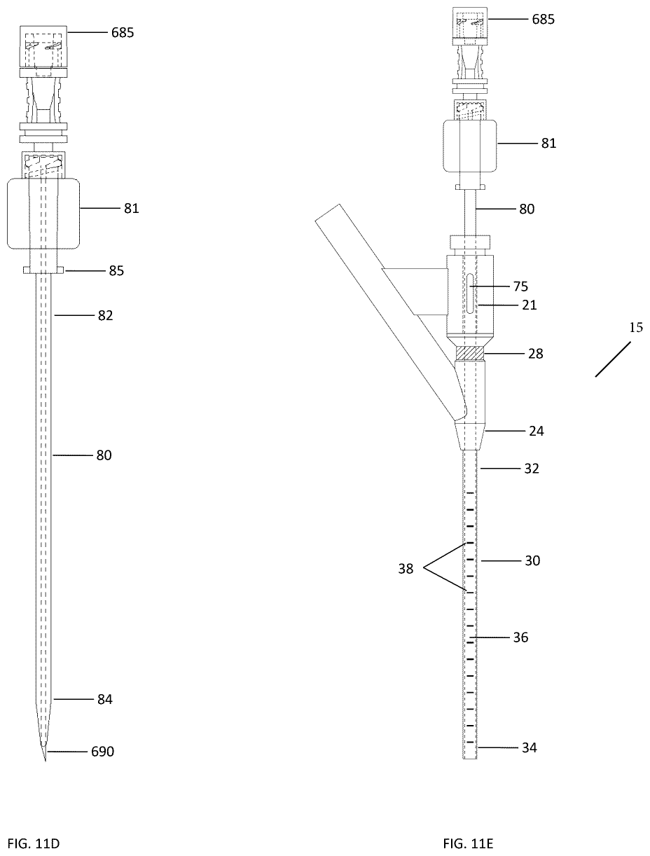

FIGS. 11a through 11e show an embodiment in which the obturator 80 further includes a needle assembly 600 operably connected to the obturator 80. A blunt or sharp tip needle assembly 600 which is longer than the obturator 80, which can be passed through the central channel 86 of the obturator 80 and is releasably secured to the obturator 80. The needle assembly 600 includes a needle sheath 620 with an inner channel 640 and needle shaft 680 with sharp puncturing distal end 690 (i.e., a solid puncturing shaft with a solid core or a sharp hollow shaft) and proximal end 685 forming a handle. The needle sheath 620 and the needle shaft 680 are releasably secured to one another through a locking mechanism 660 and 670 (i.e. a luer lock). The needle assembly 600 is then inserted into a central channel 86 of the obturator 80 and is locked in place using the connecting mechanism 695 of the needle assembly 600 to the connecting mechanism in the receptacle 88 of the obturator 80. The connected needle assembly 600 and the obturator 80 is in turn is inserted into the sheath 10 and releasably secured in place using connecting mechanisms 25 and 85. This forms the needle/obturator/sheath assembly 15. The inside channel 640 of the needle sheath 620 can accommodate a standard guidewire. In one embodiment, the entire suction evacuation needle/sheath assembly 15 (FIG. 11E) as described above can be inserted into a patient in a single step maneuver. The entire suction evacuation needle/sheath assembly 15 as described above can also be inserted over a guidewire when a guidewire has been placed through the inner channel 640 of the needle sheath 620, after removing the needle shaft 680.

### Addition of Collection Container

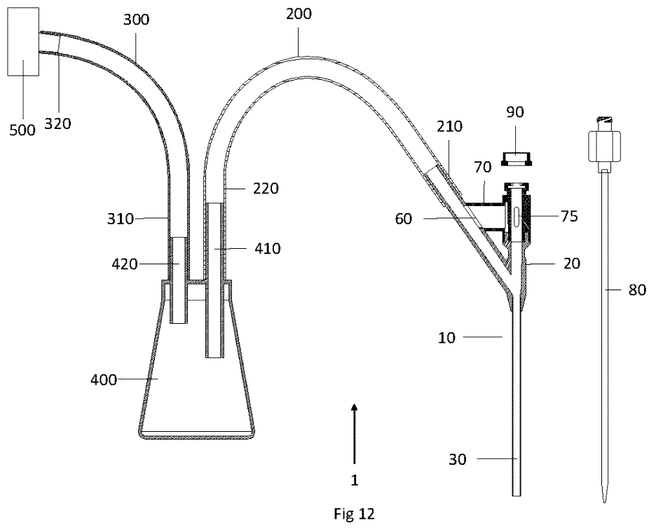

FIG. 12 shows a final embodiment of the Suction endoscopic sheath assembly with transposed pressure regulating mechanism in which a proximal end 210 of a primary tube 200 is releasably secured to the distal end 44 of the side branch 40 which contains the transposed pressure regulating mechanism 75; and the distal end 220 of the primary tube is releasably secured to ingress port 410 of the collection container 400; and the proximal end 310 of a secondary tube 300 is releasably secured to the collection container 400 egress port 420 and the distal end 320 of the secondary tube 300 to a releasably secured to a negative pressure apparatus 500.

### Methodology

The present disclosure relates to a method of using a suction sheath assembly 1 for removing one or more stones or foreign bodies from within a patient. The suction evacuation assembly includes a sheath 10, an obturator 80, a flexible seal 90, connecting tubing 200 and 300, and collection container 400, and a negative pressure apparatus (500). More specifically, the present invention discloses a method for removing a stone, a stone fragment or a foreign body from a patient using a suction evacuation assembly 1 including the steps of:
(a) providing a suction evacuation assembly 1 which includes a sheath 10 and one or more oblique side arms 40;
(b) inserting an obturator 80 into a proximal end of a sheath 10 and securing the obturator to the proximal end of the sheath 10, said sheath being formed of a proximal tube 20 and a distal tube 30, wherein the proximal tube 20 has a proximal end 22 and a distal end 24 and the distal tube 30 has a proximal end 32 and a distal end 34, wherein the distal end 24 of the proximal tube 20 is secured to the proximal end 32 of the distal tube 30 and wherein the proximal tube 20 has one or more oblique side arms 40 emanating from the outer surface of the proximal tube 20, and a pressure vent 60 located on the one or more oblique side arms 40 The pressure vent 60 is in an opening the extends through the wall of the side arm 40 and is in direct communication with the lumen 46 of the side branch. The pressure vent 60 is sealed by a channel 70. Channel 70 has a proximal end 72 and distal end 74 respective to the pressure vent 60. The proximal end 72 encases the pressure vent 60 and the distal end 74 extends to the proximal tube 20 adjacent to the proximal end 22. Channel 70 has a lumen 76 and an opening 75 located on or near the surface 21 of the proximal end 22 of the proximal tube 20 of the sheath 10 and serves as the pressure regulating mechanism. Said opening 75 does not communicate directly with the lumen 26 of the proximal tube 20. The opening 75 can be covered, partially covered, or left open to regulate the suction pressure.
(c) inserting a distal end 34 of the distal tube 30 or the distal end 59 of the deflectable tip 55 of the sheath 10 into a lumen or cavity of a patient's body containing one or more stones or foreign bodies;
(d) positioning the distal end 34 to of the distal tube or the distal end 59 of the distal tip 55 in a position in close proximity to the stones or foreign bodies;
(e) disengaging the obturator 80 from the proximal end 22 of the sheath and removing the obturator from the sheath 10;
(f) securing a flexible seal 90, if it is releasable, to the proximal end of the proximal tube;
(g) connecting the primary end 210 of a primary tube 200 to the distal end 44 of the side arms 40 that contain the pressure vent 60 and connecting the distal end end (220) of the primary tube (200) to a collection container 400;
(h) connecting the proximal end 310 of a secondary tube 300 to the collection container 400 and connecting the distal end 320 of the secondary tube 300 to a negative pressure apparatus 500;
(i) inserting a scope, which has an outer diameter smaller than the inner diameters of the lumens 26, 36, and 56 of the sheath 10, through the flexible seal 90 and into the sheath 10 then into a patient thereby forming an open channel or within the sheath 10 between the outer surface of the scope and the inner surface of the lumens 26, 36 and 56;
(j) visualizing the stone or foreign body using the scope; deflecting the deflectable tip 55, if present, at the distal end (34) of the distal tube (30) as necessary in order to visualize the stone;
(k) activating the negative pressure apparatus (500) in order to aspirate the stone or foreign bodies from the cavity if a diameter of the stone or foreign body is narrower than the inside diameter of the lumens (26, 36, 56) and the inside diameter of the side arm lumen 46;
(l) performing lithotripsy on the stone or the foreign body in order to create fragments with a decreased diameter which allow the passage of the fragments in the space between the scope and an inner surface of the lumens 26, 36, 56 and side arm lumen 46, or within the inside diameter of the sheath lumen 26, 36, 56 and the lumen (46) of the side arm (40) by withdrawing the endoscope proximal to the adjoining point 45 of the sheath 10 and the oblique side branch 40 (demarcated by the color band 28);
(m) collecting the stone, stone fragments, or foreign body in the collection container 400.

1. A suction endoscopic sheath device for removing stone and stone fragments from a patient comprising:
a straight sheath (10) comprising:
a proximal tube (20) with a lumen (26), a proximal end (22) and a distal end (24);
a first distal tube (30) with a lumen (36), a proximal end (32) and a distal end (34); wherein the distal end (24) of the proximal tube (20) is secured to the proximal end (32) of the distal tube (30); and wherein the distal tube (30) may have graduation markings to measure distance on its surface (31); wherein the distal tube may have uniform or variable flexibility;
one or more side branches (40) with a proximal end (42) and a distal end (44), said one or more side branches (40) extend away from the distal end (24) of the proximal tube (20); wherein each of the one or more side branches (40) has a lumen (46) extending from the proximal end (42) to the distal end (44) and joining with the lumen (26) of the proximal tube (20) at the distal end (24) of the proximal tube (20);
wherein at least one of the at least one side branches (40) contains a pressure vent (60) in the form of an opening extending through a wall (41) of the one or more side branches (40) and being enclosed by an airtight pressure vent channel (70);
wherein the pressure vent channel (70) extends at least partially around the proximal end (22) of the proximal tube (20) of the straight sheath (10); said pressure vent channel has at least one opening (75) located and accessible on or near the surface (21) of said proximal tube (20) such that an operator can regulate the aspiration pressure by covering, partially covering or uncovering the at least one opening (75);
an obturator (80) releasably secured to the proximal end (22) of the proximal tube (20); said obturator (86) having at least one central channel (86) said obturator inserted into the proximal end (22) of the proximal tube (20) and extending beyond the distal end (34) of the first distal tube (30); and
a flexible, water-tight seal (90) with central aperture (95) that is releasably or non-releasably secured to the proximal end (22) of the proximal tube (20).

2. The device of claim 1 wherein airtight pressure vent channel (70) extends onto the proximal tube (20) such that it partially encircles a surface (21) of the proximal tube (20); wherein said at least one opening (75) is two openings (75, 75′).

3. The device of claim 1 wherein a plug (78), or a sliding push-pull or rotating valve (77) is used to cover, partially cover, or leave open the pressure regulating mechanism (75).

4. The device of claim 1 further comprising:
a flexible, deflectable tip (55) incorporated into the distal end (34) of the first distal tube (30) of the sheath (10); wherein the flexibility of the deflectable tip may be uniform or variable.

5. The device of claim 4 further comprising a transition point (55a) at which the flexibility of the deflectable tip (55) increases.

6. The device of claim 1 wherein the first distal tube (30) of sheath (10) is releasably attached to the proximal tube (20) and there is a perforated or tearable section (35) within the first distal tube (30).

7. The device of claim 6 wherein the first distal tube (30) of sheath 10 includes two flanges (33), which an operator can grasp to tear the distal tube (30).

8. The device of claim 1 further comprising a balloon assembly (153) connected to the distal end (34) of the first distal tube (30) of the sheath (10) or the distal end (59) of the deflectable tip (55), said balloon assembly comprising:
a balloon inflation valve (154) connected to a balloon (150) via a balloon inflation channel (152) wherein passing a gas or liquid through the balloon inflation valve (154) through the balloon inflation channel (152) causes the balloon (150) to inflate.

9. The device of claim 1 further comprising:
a needle assembly (600) operably connected to the obturator (80), said needle assembly (600) comprising:
a needle sheath (620) with an inner channel (640) and needle shaft (680) secured to one another with a locking mechanism (660, 670);
wherein the needle assembly (600) is longer than the obturator (80), and passes through a central channel (88) of the obturator (80) and is secured to the obturator (80); and
wherein the needle assembly and the obturator (80) are inserted into the sheath (10).

10. The device of claim 1 further comprising a plurality of side branches (40) each with a lumen (46) that communicates with the lumen (26) of the proximal tube (20) and the lumen (36) of the first distal tube (30).

11. The device of claim 1 further comprising a plurality of accessory side arms (160) wherein each of the plurality of accessory side arms (160) has a separate lumen (166) within the sheath (10).

12. The device of claim 1 wherein a scope has an outer diameter which is smaller than an inner diameter of the lumens (26, 36) of the sheath (10) resulting in an open channel within the sheath (10) which permits passage of the stones, stone fragments, or tissue through the lumens (26, 36) of the proximal tune (20) and the first distal tube (30) and through the lumen (46) of the side branch (40).

13. The device of claim 1 further comprising a second distal tube (180) with a diameter larger than a diameter of the first distal tube (30); wherein the second distal tube (180) detachably or non-detachably encloses the first distal tube (30) concentrically and also encloses a second distal lumen (146); wherein the second distal tube (180) also contains one or more side arms (185) each with a lumen (186) that is continuous with and communicates with the second distal lumen (146).

14. The device of claim 1 further comprising:
a secondary tube (800) releasably or non-releasably secured to an outer surface (31) of the distal tube (30); and
a single or a plurality of second side arms (700) with a lumen or a plurality of lumens (760) that is/are continuous with and communicate(s) with an accessory channel or plurality of accessory channels (860) of the secondary tube (800).

15. A suction endoscopic sheath assembly for removing stone and stone fragments from a patient comprising:
a straight sheath (10) comprising:
a proximal tube (20) with a lumen (26), a proximal end (22) and a distal end (24);
a first distal tube (30) with a lumen (36), a proximal end (32) and a distal end (34); wherein the distal end (24) of the proximal tube (20) is secured to the proximal end (32) of the first distal tube (30); and wherein the first distal tube (30) may have graduation markings to measure distance on its surface (31);
wherein the distal tube (30) may have uniform or variable flexibility;
one or more side branches (40) with a proximal end (42) extending away from the distal end (24) of the proximal tube (20); wherein each of the one or more side branches (40) has a lumen (46) extending from the proximal end (42) to a distal end (44) and joining with the lumen (26) of the proximal tube (20) at the distal end (24) of the proximal tube (20); wherein at least one of the at least one side branches (40) contains a pressure vent (60) in the form of an opening extending through a wall (41) of the one or more side branches (40) and being enclosed by an airtight pressure vent channel (70);
wherein the pressure vent channel (70) extends toward the proximal tube (20) of the straight sheath (10) and has at least one opening (75) located and accessible on or near the surface (21) of said proximal tube (20) such that an operator can regulate the aspiration pressure by partially or completely covering and uncovering the opening (75);
an obturator (80) with one or more central channels (86, 86′) inserted into the proximal end (22) of the proximal tube (20); said one or more central channels extending beyond the distal end (34) of the distal tube (30), wherein the proximal end (82) of the obturator is releasably secured to the proximal end (22) of the proximal tube (20);
a flexible water-tight seal (90) with central aperture (95) that is releasably or non-releasably secured to the proximal end (22) of the proximal tube (20);
a primary end (210) of a primary tube (200) releasably secured to a distal end (44) of the side branch (40) which contains the transposed pressure regulating mechanism (75); and
a distal end (220) of the primary tube (200) releasably secured to a collection container (400); and the proximal end (310) of a secondary tube (300) is releasably secured to the collection container (400) and the distal end (320) of the secondary tube (300) is releasably secured to a negative pressure apparatus (500).

16. The assembly of claim 15 further comprising a flexible, deflectable tip (55) incorporated into the distal end (34) of the distal tube (30), which will enable to operator to adjust the direction of the flexible, deflectable tip (55) passively by using a flexible instrument such as endoscope or actively by using inherent cables or other means.

17. The assembly of claim 15 further comprising a balloon assembly (153) connected to the distal end (34) of the first distal tube (30) of the sheath (10), said balloon assembly comprising:
a balloon inflation valve (154) connected to a balloon (150) via a balloon inflation channel (152) wherein passing a gas or liquid through the balloon inflation valve (154) through the balloon inflation channel (152) causes the balloon (150) to inflate.

18. The assembly of 15 wherein each of the one or more side branches (40) has a separate channel (46) within the sheath (10).

19. The assembly of claim 15 further comprising a second distal tube (180) with a diameter larger than a diameter of the first distal tube (30); wherein the second distal tube (180) detachably or non-detachably encloses the first distal tube (30) concentrically and also encloses a second distal lumen (146); wherein the second distal tube (180) contains one or more side arms (185) each with a lumen (186) that is continuous with and communicates with the second distal lumen (146).

20. The assembly of claim 15 further comprising a second distal tube (800) releasably or non-releasably secured to the first distal tube (30) or the proximal tube (20) non-concentrically; wherein the second distal tube (800) also contains one or more side arms (700) each with a lumen (760) that is continuous with and communicates with a lumen (860) of the second distal tube (800).

21. The assembly of claim 15 further comprising:
a scope with an outer diameter which is smaller than an inner diameter of the lumens (26, 36) of the sheath (10) resulting in an open channel within the sheath (10) which permits passage of the stones, stone fragments, or tissue through the lumens (26, 36) of the proximal tune (20) and the first distal tube (30) and through the lumen (46) of the side branch (40).

22. (canceled)

23. (canceled)

24. (canceled)

25. (canceled)

26. (canceled)

27. (canceled)

28. (canceled)

29. The device of claim 4 wherein the flexibility of the deflectable tip (55) may increase or decrease as one moves from the proximal end (57) to the distal end (59) of said deflectable tip (55).

30. The assembly of claim 16 wherein the flexibility of the deflectable tip (55) may increase or decrease as one moves from the proximal end (57) to the distal end (59) of said deflectable tip (55).
